(19) United States
(12) Patent Application Publication
(10) Pub. No.: US 2011/0081307 A1
(43) Pub. Date: Apr. 7, 2011

(54) KIT COMPRISING AN ALGINATE AND A COMPLEXING AGENT IN THE FORM OF A WATER-SOLUBLE SALT

(75) Inventor: Olivier Raineau, Paris (FR)

(73) Assignee: L'OREAL, Paris (FR)

(21) Appl. No.: 12/808,965

(22) PCT Filed: Dec. 16, 2008
(86) PCT No.: PCT/EP08/67656

§ 371 Date: Dec. 6, 2010

Related U.S. Application Data
(60) Provisional application No. 61/021,045, filed on Jan. 15, 2008.

(30) Foreign Application Priority Data
Dec. 21, 2007 (FR) 0760247

(51) Int. Cl. A61K 8/73; A61Q 1/00; A61Q 1/02; A61Q 1/04; A61Q 1/08; A61Q 1/10; A61Q 3/00; A61Q 3/02; A61Q 3/04; A61K 8/44; A61K 8/55; A61Q 1/14; A61Q 5/02

(52) U.S. Cl. 424/61; 424/70.13; 424/63; 424/64; 514/54; 510/118; 510/136; 510/119; 132/200

(57) ABSTRACT

The present patent application relates to a keratin material coating kit comprising: a first composition comprising an aqueous phase, at least one alginic acid-based compound A and at least one compound B in the form of a water-soluble salt, the said compound being capable of releasing a gas on contact with an acid having a pKa ranging from 1 to 6, and a second composition comprising at least one acid having a pKa ranging from 1 to 6 and at least one agent C for complexing the alginic acid-based compound, the said agent being in the form of a water-soluble salt.

The present patent application relates to the field of making up or caring for keratin materials, in particular the skin, the nails, the lips and, especially, the eyelashes, and preferably makeup compositions.

Each composition may be a loose or compacted powder, a foundation, a makeup rouge, an eyeshadow, a concealer product, a blusher, a lipstick, a lip balm, a lip gloss, a lip pencil, an eye pencil, a mascara, an eyeliner, a nail varnish, a body makeup product or a skin colouring product.

Consumers are looking for cosmetic products that can increase the perception of volume of the keratin materials they wish to make up. In particular, a charging or volumizing effect on the eyelashes is desired for massacres, a fleshy effect is sought for lip glosses and lipsticks, and facial modelling properties and masking of skin imperfections (wrinkles, fine lines, pigmentation defects, loss of lip colour, or couperosis) are demanded by foundation and lipstick users.

In addition, these cosmetic compositions must have good staying power over time, so that the aesthetic effect obtained is maintained.

At the present time, the remodelling and increase in volume of certain parts of the face or body is obtained by injecting substances such as silicone gels. This type of remodelling is generally performed under local anaesthesia. In addition, this type of remodelling is long, laborious and expensive.

It is moreover known that a volumizing effect may be produced by applying a light shade and a dark shade next to each other, the light shade being applied to the area that it is desired to highlight. Obtaining this effect conventionally requires the use of two different compositions and is dependent on the skill of the person applying them. Furthermore, this technique is difficult to perform for making up the lips.

It is also known practice, for example from documents EP 0 953 330, WO 01/51015 or EP 1 382 323, to use pigments with an optical effect (goniochromatic pigments or interference pigments) to modify the perception of volume of the part of the body to which the composition is applied, depending on the angle of observation or the angle of incidence of the light.

In the case of massacres, compositions with a high solids content are generally used, in order to provide the keratin materials with matter, but these compositions have a high consistency, which is incompatible with homogeneous application to the eyelashes. Patent application US 2006/0 140 895 also discloses kits of compositions with a swelling effect comprising polymers having a high water-absorbing capacity.

However, obtaining the swelling effect requires the provision of a large amount of water, and the swelling obtained is not long lasting due to the evaporation of the water. Document US 2004/0 126 345 also discloses gels with delayed expansion based on (meth)acrylate polymer, which are aimed at obtaining a volumizing effect on keratin materials.

It is thus sought to obtain a keratin material coating composition that can produce on the keratin materials a film that has good staying power and/or good water resistance and that produces a volumizing effect on the keratin materials, and that can be easily removed.

A first subject of the present patent application is a keratin material coating kit comprising:
a first composition comprising an aqueous phase, at least one alginic acid-based compound A and at least one compound B in the form of a water-soluble salt, the said compound being capable of releasing a gas on contact with an acid having a pKa ranging from 1 to 6, and
a second composition comprising at least one acid having a pKa ranging from 1 to 6 and at least one agent C for complexing the alginic acid-based compound, the said agent being in the form of a water-soluble salt.

A second subject of the present patent application is a cosmetic process for coating keratin materials, which consists in applying to the said keratin materials:
at least one coat of at least one first composition comprising an aqueous phase, at least one alginic acid-based compound A and at least one compound B in the form of a water-soluble salt, the said compound B being capable of releasing a gas on contact with an acid having a pKa ranging from 1 to 6, and then
at least one coat of at least one second composition comprising at least one acid having a pKa ranging from 1 to 6 and at least one agent C for complexing the alginic acid-based compound, the said agent being in the form of a water-soluble salt.

The terms “first composition” and “second composition” do not in any way condition the order of application of the said compositions to the keratin materials.

Preferably, at least one coat of the first composition and then at least one coat of the second composition are applied to the keratin materials.

Moreover, the coating or film formed after applying the first and second compositions described above may be readily removed using a composition comprising an agent for chelating the complexing agent.

Accordingly, according to another aspect, a subject of the present invention is also a process for removing and/or cleansing away makeup films formed by application, to keratin materials, of a first composition comprising an aqueous phase, at least one alginic acid-based compound and at least one compound B in the form of a water-soluble salt, the said compound being capable of releasing a gas on contact with an acid having a pKa ranging from 1 to 6, and of a second composition comprising at least one acid having a pKa ranging from 1 to 6 and at least one agent C for complexing the alginic acid-based compound, the said agent being in the form of a water-soluble salt, the process comprising at least one step of applying to the said makeup films a makeup-removing and/or cleansing composition comprising, in a physiologically acceptable medium, an aqueous phase and at least one sequestrant for the said complexing agent.

Other characteristics, properties and advantages of the present invention will emerge more clearly on reading the description and the examples that follow.

### Alginic Acid-Based Compound A

The term “alginic acid-based compound” in particular means alginic acid, alginic acid derivatives and alginic acid salts (alginates) or salts of the said derivatives. Preferably, the alginic acid-based compound is water-soluble.

Alginic acid, a natural substance obtained from brown algae or from certain bacteria, is a polyuronic acid composed of two uronic acids linked together via 1,4-glycoside bonds: β-D-mannuronic acid (M) and α-L-glucuronic acid (G).

Alginic acid is capable of forming water-soluble salts (alginates) with alkali metals such as sodium, potassium or lithium, cations of lower amines and of substituted ammonium such as methylamine, ethanolamine, diethanolamine or triethanolamine. These alginates are water-soluble in aqueous medium at pH 4 but dissociate into alginic acid at a pH below 4.

These alginates are capable of crosslinking together in the presence of complexing agents, by forming ionic bonds between the said complexing agents and the negatively charged group of the residue G. The formation of multiple crosslinks between several alginate molecules leads to the formation of a matrix that forms an insoluble gel in water.

Alginic acid-based compounds having a weight-average molecular mass ranging from 10 000 to 1 000 000, preferably from 15 000 to 500 000 and better still from 20 000 to 250 000 are preferably used.

Preferably, the first composition has a pH ranging from 5 to 10.

According to one preferred embodiment, the alginic acid-based compound is a sodium or potassium alginate.

The alginic acid-based compound may be present in a content ranging from 0.1% to 30% by weight, preferably from 0.5% to 20% by weight and better still from 1% to 10% by weight relative to the total weight of the composition comprising it.

Compound B in the Form of a Water-Soluble Salt Capable of Releasing a Gas on Contact with Acid

The compound B present in the first composition in the form of a water-soluble salt is capable of releasing a gas on contact with an acid having a pKa ranging from 1 to 6. It may be chosen in particular from polyelectrolytes and ions, in the form of a water-insoluble salt.

The complexing agent may be chosen in particular from alkali metal carbonates or hydrogen carbonates (also called bicarbonates), such as the salts of alkali metals such as Na or K and of CO₃²⁻ or HCO₃⁻.

Compounds B which lead, after reaction with the acid having a pKa ranging from 1 to 6 present in the second composition, to the release of CO₂, for instance carbonates of alkali metal cations, in particular sodium carbonate or bicarbonate, are preferably used.

During the application of the second composition, the acid present in this composition entrains the dissociation of compound B and an evolution of gas takes place. In parallel, the complexing agent C in the form of a water-soluble salt becomes complexed with the alginic acid-based compound by formation of an ionic bond. When compound B is an alkali metal carbonate, the application of the acid having a pKa ranging from 1 to 6 contained in the second composition entrains an evolution of CO₂ gas, the gas bubbles being trapped in the network/gel formed by the alginate complexed with the complexing agent, which leads to an increase in the volume of the deposit formed on the eyelashes and to the production of a volumizing effect on the eyelashes.

The compound(s) B in the form of water-soluble salts may be present in a content ranging from 0.01% to 30% by weight, preferably from 0.02% to 15% by weight and better still from 0.05% to 5% by weight relative to the total weight of the first composition.

### Complexing Agent C in Water-Soluble Form

The complexing agent is capable of becoming complexed with the alginic acid-based compound by formation of an ionic bond. This complexation is reversible.

The water-soluble complexing agent may be chosen in particular from polyelectrolytes and multivalent ions and salts thereof, in particular the salts of multivalent cations or of polycations. It is preferably in water-soluble form in the second composition.

The multivalent ions may in particular be cations such as polycations or multivalent cations, preferably having a valency ranging from 2 to 4 and more preferably divalent cations, in particular calcium, magnesium, aluminium, barium, zinc, nickel, copper (+II) and manganese cations, and mixtures thereof. Mention may be made especially of the chlorides or sulfates of the cations mentioned above and in particular chlorides such as calcium chloride.

A polyelectrolyte that may be mentioned is polyethylene amine.

The complexing agent(s) may be present in a content ranging from 0.01% to 30% by weight, preferably from 0.02% to 15% by weight and better still from 0.05% to 5% by weight relative to the total weight of the second composition.

The rate of reaction between the polysaccharide and the complexing agent may be modulated by modifying the pH and/or temperature conditions of each composition, or alternatively by adding a compound that can accelerate or slow down the reaction between the polysaccharide and the complexing agent, for instance sodium phosphate, which can slow down the crosslinking of the alginic acid-based derivative.

### Acid

The acid having a pKa ranging from 1 to 6, and preferably from 3 to 6, present in the second composition may be chosen in particular from sulfuric acids and carboxylic acids, and mixtures thereof, preferably from carboxylic acids such as propanoic acid, butanoic acid, acetic acid, lactic acid, citric acid or tartaric acid.

Tartaric acid or citric acid is preferably used.

The acid having a pKa ranging from 1 to 6 may be present in the second composition in a content ranging from 0.0001% to 15% by weight and preferably from 0.001% to 10% by weight relative to the total weight of the second composition comprising it.

Aqueous Phase

The first composition according to the invention comprises an aqueous phase, which may form the continuous phase of the composition.

The term “composition with a continuous aqueous phase” means that the composition has a conductivity, measured at 25° C., of greater than 23 μS/cm (microSiemens/cm), the conductivity being measured, for example, using an MPC227 conductimeter from Mettler Toledo and an Inlab730 conductivity measuring cell. The measuring cell is immersed in the composition so as to remove any air bubbles liable to form between the two electrodes of the cell. The conductivity reading is taken once the conductimeter value has stabilized. A mean is determined over at least three successive measurements.

The aqueous phase comprises water and/or at least one water-soluble solvent.

In the present invention, the term “water-soluble solvent” denotes a compound that is liquid at room temperature and water-miscible (miscibility in water of greater than 50% by weight at 25° C. and atmospheric pressure).

The water-soluble solvents that may be used in the compositions according to the invention may also be volatile.

Among the water-soluble solvents that may be used in the compositions according to the invention, mention may be made especially of lower monoalcohols containing from 1 to 5 carbon atoms, such as ethanol and isopropanol, glycols containing from 2 to 8 carbon atoms, such as ethylene glycol, propylene glycol, 1,3-butylene glycol and dipropylene glycol, C₃-C₄ ketones and C₂-C₄ aldehydes.

The aqueous phase (water and optionally the water-miscible solvent) is generally present in the composition according to the present patent application in a content ranging from 1% to 95% by weight, preferably ranging from 3% to 80% by weight and preferentially ranging from 5% to 60% by weight relative to the total weight of the composition.

The second composition may comprise an aqueous phase as described above.

Emulsifying System

The first and/or the second composition may comprise an emulsifying system comprising one or more surfactants.

According to the invention, an emulsifier appropriately chosen to obtain an oil-in-water emulsion is generally used. In particular, an emulsifier having at 25° C. an HLB (hydrophilic-lipophilic balance), in the Griffin sense, of greater than or equal to 8 may be used.

The HLB value according to Griffin is defined in J. Soc. Cosm. Chem. 1954 (volume 5), pages 249-256.

These surfactants may be chosen from nonionic, anionic, cationic and amphoteric surfactants or emulsifying surfactants. Reference may be made to the document “Encyclopaedia of Chemical Technology, Kirk-Othmer”, volume 22, pp. 333-432, 3rd edition, 1979, Wiley, for the definition of the properties and (emulsifying) functions of surfactants, in particular pp. 347-377 of this reference, for anionic, amphoteric and nonionic surfactants.

The surfactants preferably used in the composition according to the invention are chosen from:
a) nonionic surfactants with an HLB of greater than or equal to 8 at 25° C., used alone or as a mixture; mention may be made especially of:
   oxyethylenated and/or oxypropylenated ethers (which may comprise from 1 to 150 oxyethylene and/or oxypropylene groups) of glycerol;
   oxyethylenated and/or oxypropylenated ethers (which may comprise from 1 to 150 oxyethylene and/or oxypropylene groups) of fatty alcohols (especially of C₈-C₂₄ and preferably C₁₂-C₁₈ alcohol), such as oxyethylenated stearyl alcohol ether containing 20 oxyethylene groups (CTFA name “Steareth-20”) such as Brij 78 sold by the company Uniqema, oxyethylenated cetearyl alcohol ether containing 30 oxyethylene groups (CTFA name “Ceteareth-30”) and the oxyethylenated ether of the mixture of C₁₂-C₁₅ fatty alcohols comprising 7 oxyethylene groups (CTFA name “C12-15 Pareth-7” sold under the name Neodol 25-7® by Shell Chemicals);
   fatty acid esters (especially of a C₈-C₂₄ and preferably C₁₆-C₂₂ acid) of polyethylene glycol (which may comprise from 1 to 150 ethylene glycol units), such as PEG-50 stearate and PEG-40 monostearate sold under the name Myrj 52P® by the company ICI Uniqema;
   fatty acid esters (especially of a C₈-C₂₄ and preferably C₁₆-C₂₂ acid) of oxyethylenated and/or oxypropylenated glyceryl ethers (which may comprise from 1 to 150 oxyethylene and/or oxypropylene groups), for instance PEG-200 glyceryl monostearate sold under the name Simulsol 220 TM® by the company SEPPIC; glyceryl stearate polyethoxylated with 30 ethylene oxide groups, for instance the product Tagat S® sold by the company Goldschmidt, glyceryl oleate polyethoxylated with 30 ethylene oxide groups, for instance the product Tagat O® sold by the company Goldschmidt, glyceryl cocoate polyethoxylated with 30 ethylene oxide groups, for instance the product Varionic LI 13® sold by the company Sherex, glyceryl isostearate polyethoxylated with 30 ethylene oxide groups, for instance the product Tagat L® sold by the company Goldschmidt, and glyceryl laurate polyethoxylated with 30 ethylene oxide groups, for instance the product Tagat I® from the company Goldschmidt;
   fatty acid esters (especially of a C₈-C₂₄ and preferably C₁₆-C₂₂ acid) of oxyethylenated and/or oxypropylenated sorbitol ethers (which may comprise from 1 to 150 oxyethylene and/or oxypropylene groups), for instance polysorbate 60 sold under the name Tween 60® by the company Uniqema;
   dimethicone copolyol, such as the product sold under the name Q2-5220® by the company Dow Corning;
   dimethicone copolyol benzoate (Finsolv SLB 101® and 201® by the company Finetex);
   copolymers of propylene oxide and of ethylene oxide, also known as EO/PO polycondensates;
and mixtures thereof.

The EO/PO polycondensates are more particularly copolymers consisting of polyethylene glycol and polypropylene glycol blocks, for instance polyethylene glycol/polypropylene glycol/polyethylene glycol triblock polycondensates. These triblock polycondensates have, for example, the following chemical structure:

H—(O—CH₂—CH₂)ₐ—(O—CH(CH₃)—CH₂)_{b}—(O—CH₂—CH₂)ₐ—OH, in which formula a ranges from 2 to 120 and b ranges from 1 to 100.

The EO/PO polycondensate preferably has a weight-average molecular weight ranging from 1000 to 15 000 and better still ranging from 2000 to 13 000. Advantageously, the said EO/PO polycondensate has a cloud point, at 10 g/l in distilled water, of greater than or equal to 20° C. and preferably greater than or equal to 60° C. The cloud point is measured according to ISO standard 1065. As EO/PO polycondensates that may be used according to the invention, mention may be made of the polyethylene glycol/polypropylene glycol/poly-ethylene glycol triblock polycondensates sold under the name Synperonic®, for instance Synperonic PE/L44® and Synperonic PE/F127®, by the company ICI.
b) nonionic surfactants with an HLB of less than 8 at 25° C., optionally combined with one or more nonionic surfactants with an HLB of greater than 8 at 25° C., such as those mentioned above, such as:
   saccharide esters and ethers, such as sucrose stearate, sucrose cocoate and sorbitan stearate, and mixtures thereof, for instance Arlatone 2121® sold by the company ICI or Span 65V from the company Uniqema;
   fatty acid esters (especially of a C₈-C₂₄ and preferably C₁₆-C₂₂ acid) of polyols, especially of glycerol or of sorbitol, such as glyceryl stearate, glyceryl stearate such as the product sold under the name Tegin M® by the company Goldschmidt, glyceryl laurate such as the product sold under the name Imwitor 312® by the company Hüls, polyglyceryl-2 stearate, sorbitan tristearate or glyceryl ricinoleate;
   oxyethylenated and/or oxypropylenated ethers such as the oxyethylenated ether of stearyl alcohol containing two oxyethylene groups (CTFA name “Steareth-2”), such as Brij 72 sold by the company Uniqema;
   the mixture of cyclomethicone/dimethicone copolyol sold under the name Q2-3225C® by the company Dow Corning,
c) anionic surfactants such as:
   polyoxyethylenated fatty acid salts, especially those derived from amines or alkali metal salts, and mixtures thereof;
   phosphoric esters and salts thereof, such as “DEA oleth-10 phosphate” (Crodafos N 10N from the company Croda) or monocetyl monopotassium phosphate (Amphisol K from Givaudan or Arlatone MAP 160K from the company Uniqema);
   sulfosuccinates such as “Disodium PEG-5 citrate lauryl sulfosuccinate” and “Disodium ricinoleamido MEA sulfosuccinate”;
   alkyl ether sulfates, such as sodium lauryl ether sulfate;
   isethionates;
   acylglutamates such as “Disodium hydrogenated tallow glutamate” (Amisoft HS-21 R® sold by the company Ajinomoto), and mixtures thereof.

Cationic surfactants that may especially be mentioned as representations include:
alkylimidazolidiniums such as isostearylethyl-imidonium ethosulfate,
ammonium salts such as N,N,N-trimethyl-1-docosanaminium chloride (behentrimonium chloride).

The compositions according to the invention may also contain one or more amphoteric surfactants, for instance N-acylamino acids such as N-alkylaminoacetates and disodium cocoamphodiacetate, and amine oxides such as stearamine oxide, or alternatively silicone surfactants, for instance dimethicone copolyol phosphates such as the product sold under the name Pecosil PS 100® by the company Phoenix Chemical.

According to one embodiment, the emulsifying system of the first composition comprises at least one C₁₀-C₃₀ alkyl phosphate.

According to one embodiment, the emulsifying system of the composition comprises at least one C₁₀-C₃₀ alkyl phosphate and at least one ether of a C₈-C₂₄ fatty alcohol and of polyethylene glycol, the said ether comprising from 1 to 19 ethylene glycol units and having an HLB value <8 at 25° C.

This emulsifying system may also comprise at least one ether of a C₈-C₂₄ fatty alcohol and of polyethylene glycol, the said ether comprising from 20 to 1000 ethylene glycol units and having an HLB value >8 at 25° C. and at least one fatty alcohol comprising from 10 to 30 carbon atoms, such as cetyl alcohol.

According to one embodiment, the emulsifying system of the first composition comprises at least one surfactant chosen from:
glutamic acid derivatives and salts thereof such as acyl glutamic acids (INCI name: acyl glutamic acid), and salts thereof (glutamates), for instance lauroyl glutamic acid, cocoyl glutamic acid, sodium stearoyl glutamate, potassium lauroyl glutamate, potassium cocoyl glutamate, sodium olivoyl glutamate or disodium hydrogenated tallow glutamate, and mixtures thereof,
sarcosine derivatives of formula:

CH₃—N(R)—CH₂—COOH

in which R is an acyl group O═CR′, R′ being a linear or branched, saturated or unsaturated hydrocarbon-based chain containing from 10 to 30 carbon atoms and preferably from 12 to 22 carbon atoms, or a cosmetically acceptable salt thereof, in particular myristoyl sarcosine derivatives, palmitoyl sarcosine derivatives, oleoyl sarcosine derivatives, stearoyl sarcosine derivatives, preferably stearoyl and palmitoyl sarcosine derivatives, or cosmetically acceptable salts thereof (sarcosinates). Mention may be made especially of sodium palmitoyl sarcosinate, magnesium palmitoyl sarcosinate, myristoyl sarcosine and stearoyl sarcosine, and mixtures thereof.

According to one embodiment, the composition also comprises a co-surfactant chosen from fatty alcohols, preferably containing from 10 to 30 carbon atoms. The expression “fatty alcohol containing from 10 to 30 carbon atoms” means any saturated or unsaturated, branched or unbranched pure fatty alcohol containing from 10 to 30 carbon atoms.

A fatty alcohol containing from 10 to 26 carbon atoms, better still from 10 to 24 carbon atoms and even better still from 14 to 22 carbon atoms is preferably used.

As fatty alcohols that may be used in the composition, mention may be made especially of lauryl alcohol, myristyl alcohol, cetyl alcohol, stearyl alcohol, oleyl alcohol, cetearyl alcohol (mixture of cetyl alcohol and stearyl alcohol), behenyl alcohol and erucyl alcohol, and mixtures thereof. Cetyl alcohol is preferably used.

Such fatty alcohols are especially sold under the name Nafol by the company Sasol.

The fatty alcohol may be present in a content ranging from 0.2% to 20% by weight and preferably from 0.3% to 10% by weight relative to the total weight of the composition.

According to one embodiment, the main surfactant system of the first and/or second composition according to the invention comprises less than 1% and preferably less than 0.5% by weight of triethanolamine, and better still is free of triethanolamine.

According to one variant, the first and/or second composition comprises less than 1% by weight and preferably less than 0.5% by weight of triethanolamine stearate, and better still is free of triethanolamine stearate.

In the composition in accordance with the invention, the total content of surfactants may range from 1% to 30% by weight, preferably from 1% to 20% and better still from 2% to 15% by weight relative to the total weight of the composition comprising them.

Wax(es)

The first and/or second composition according to the present patent application may comprise at least one wax.

For the purposes of the present invention, the term “wax” means a lipophilic compound, which is solid at room temperature (25° C.), with a reversible solid/liquid change of state, which has a melting point of greater than or equal to 30° C., which may be up to 120° C.

The melting point of the wax may be measured using a differential scanning calorimeter (DSC), for example the calorimeter sold under the name DSC 30 by the company Mettler.

The waxes may be hydrocarbon-based waxes, fluoro waxes and/or silicone waxes and may be of plant, mineral, animal and/or synthetic origin. In particular, the waxes have a melting point of greater than 25° C. and better still greater than 45° C.

The wax may be present in a content ranging from 0.1% to 50% by weight, better still from 1% to 40% and even better still from 5% to 30% by weight relative to the total weight of the composition.

Hydrocarbon-based waxes, for instance beeswax, lanolin wax or Chinese insect wax; rice wax, carnauba wax, candelilla wax, ouricury wax, esparto grass wax, cork fibre wax, sugarcane wax, Japan wax and sumach wax; montan wax, microcrystalline waxes, paraffins and ozokerite; polyethylene waxes, the waxes obtained by Fisher-Tropsch synthesis and waxy copolymers, and also esters thereof, may especially be used.

Mention may also be made of waxes obtained by catalytic hydrogenation of animal or plant oils containing linear or branched C₈-C₃₂ fatty chains.

Among these waxes that may especially be mentioned are hydrogenated jojoba oil, isomerized jojoba oil such as the trans-isomerized partially hydrogenated jojoba oil manufactured or sold by the company Desert Whale under the commercial reference Iso-Jojoba-50®, hydrogenated sunflower oil, hydrogenated castor oil, hydrogenated coconut oil, hydrogenated lanolin oil and bis(1,1,1-trimethylolpropane) tetrastearate sold under the name Hest 2T-4S by the company Heterene, bis(1,1,1-trimethylolpropane) tetrabehenate sold under the name Hest 2T-4B by the company Heterene.

Mention may also be made of silicone waxes, for instance alkyl or alkoxy dimethicones containing from 16 to 45 carbon atoms, and fluoro waxes.

The wax obtained by hydrogenation of olive oil esterified with stearyl alcohol, sold under the name Phytowax Olive 18L57 or else the waxes obtained by hydrogenation of castor oil esterified with cetyl alcohol sold under the names Phytowax ricin 16L64 and 22L73 by the company Sophim may also be used. Such waxes are described in patent application FR-A-2 792 190.

According to one particular embodiment, the compositions in accordance with the invention may comprise at least one “tacky” wax, i.e. a wax with a tack of greater than or equal to 0.7 N.s and a hardness of less than or equal to 3.5 MPa.

The use of a tacky wax may especially allow the production of a cosmetic composition that is easy to apply to the eyelashes, that attaches well to the eyelashes and that leads to the formation of a smooth, uniform and thickening makeup.

The tacky wax used may especially have a tack ranging from 0.7 N.s to 30 N.s, in particular greater than or equal to 1 N.s, especially ranging from 1 N.s to 20N.s, in particular greater than or equal to 2 N.s, especially ranging from 2 N.s to 10 N.s and in particular ranging from 2 N.s to 5 N.s.

The tack of the wax is determined by measuring the change in force (compression force or stretching force) as a function of time, at 20° C., using the texturometer sold under the name TA-TX2i® by the company Rheo, equipped with a conical acrylic polymer spindle forming an angle of 45°.

The measuring protocol is as follows:

The wax is melted at a temperature equal to the melting point of the wax +10° C. The molten wax is poured into a container 25 mm in diameter and 20 mm deep. The wax is recrystallized at room temperature (25° C.) for 24 hours such that the surface of the wax is flat and smooth, and the wax is then stored for at least 1 hour at 20° C. before measuring the tack.

The texturometer spindle is displaced at a speed of 0.5 mm/s, then penetrates the wax to a penetration depth of 2 mm. When the spindle has penetrated the wax to a depth of 2 mm, the spindle is held still for 1 second (corresponding to the relaxation time) and is then withdrawn at a speed of 0.5 mm/s.

During the relaxation time, the force (compression force) decreases greatly until it becomes zero, and then, during the withdrawal of the spindle, the force (stretching force) becomes negative and then rises again to the value 0. The tack corresponds to the integral of the curve of the force as a function of time for the part of the curve corresponding to negative values of the force (stretching force). The tack value is expressed in N.s.

The tacky wax that may be used generally has a hardness of less than or equal to 3.5 MPa, in particular ranging from 0.01 MPa to 3.5 MPa, especially ranging from 0.05 MPa to 3 MPa or even ranging from 0.1 MPa to 2.5 MPa.

The hardness is measured according to the protocol described previously.

A tacky wax that may be used is a C₂₀-C₄₀ alkyl (hydroxystearyloxy)stearate (the alkyl group containing from 20 to 40 carbon atoms), alone or as a mixture, in particular a C₂₀-C₄₀ alkyl 12-(12′-hydroxystearyloxy)-stearate, of formula (II):

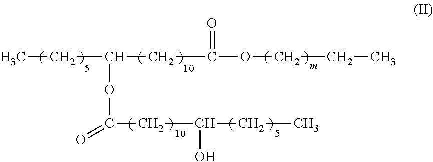

in which m is an integer ranging from 18 to 38, or a mixture of compounds of formula (II).

Such a wax is especially sold under the names Kester Wax K 82 P® and Kester Wax K 80 P® by the company Koster Keunen.

The waxes mentioned above generally have a starting melting point of less than 45° C.

The microcrystalline wax sold under the reference SP18 by the company Strahl & Pitsch, which has a hardness of about 0.46 MPa and a tack value of about 1 N.s, may also be used.

The wax(es) may be in the form of an aqueous microdispersion of wax. The expression “aqueous microdispersion of wax” means an aqueous dispersion of wax particles in which the size of the said wax particles is less than or equal to about 1 μm.

Wax microdispersions are stable dispersions of colloidal wax particles, and are described especially in “Microemulsions Theory and Practice”, L. M. Prince Ed., Academic Press (1977) pages 21-32.

In particular, these wax microdispersions may be obtained by melting the wax in the presence of a surfactant, and optionally of a portion of water, followed by gradual addition of hot water with stirring. The intermediate formation of an emulsion of the water-in-oil type is observed, followed by a phase inversion, with final production of a microemulsion of the oil-in-water type. On cooling, a stable microdispersion of solid wax colloidal particles is obtained.

The wax microdispersions may also be obtained by stirring the mixture of wax, surfactant and water using stirring means such as ultrasound, high-pressure homogenizers or turbomixers.

The particles of the wax microdispersion preferably have mean sizes of less than 1 μm (especially ranging from 0.02 μm to 0.99 μm) and preferably less than 0.5 μm (especially ranging from 0.06 μm to 0.5 μm).

These particles consist essentially of a wax or a mixture of waxes. However, they may comprise a small proportion of oily and/or pasty fatty additives, a surfactant and/or a common liposoluble additive/active agent.

The compositions according to the present patent application may also contain at least one hydrophilic or lipophilic film-forming polymer.

In the present patent application, the term “film-forming polymer” means a polymer that is capable, by itself or in the presence of an auxiliary film-forming agent, of forming a macroscopically continuous film that adheres to the eyelashes, and preferably a cohesive film, and better still a film whose cohesion and mechanical properties are such that said film can be isolated and manipulated in isolation, for example when said film is made by pouring onto a non-stick surface, for instance a Teflon-coated or silicone-coated surface.

In general, the content of “film-forming polymer” in the compositions according to the present patent application ranges from 0.1% to 40%, preferably from 0.5% to 30% and better still from 1% to 20% by weight relative to the total weight of the composition.

The hydrophilic film-forming polymer may be a water-soluble polymer or may be in dispersion in an aqueous medium.

Among the film-forming polymers that may be used in the composition of the present invention, mention may be made of synthetic polymers, of free-radical type or of polycondensates type, and polymers of natural origin, and mixtures thereof.

Examples of water-soluble film-forming polymers that may be mentioned include:
proteins, for instance proteins of plant origin such as wheat or soybean proteins; proteins of animal origin such as keratins, for example keratin hydrolysates and sulfonic keratins;
cellulose polymers such as hydroxyethylcellulose, hydroxypropylcellulose, methylcellulose, ethylhydroxy-ethylcellulose and carboxymethylcellulose, and also quaternized cellulose derivatives;
acrylic polymers or copolymers, such as polyacrylates or polymethacrylates;
vinyl polymers, for instance polyvinylpyrrolidones, copolymers of methyl vinyl ether and of maleic anhydride, the copolymer of vinyl acetate and of crotonic acid, copolymers of vinylpyrrolidone and of vinyl acetate; copolymers of vinylpyrrolidone and of caprolactam; polyvinyl alcohol;
anionic, cationic, amphoteric or nonionic chitin or chitosan polymers;
gum arabics, guar gum, xanthan derivatives and karaya gum;
alginates and carrageenans;
   glycoaminoglycans, and hyaluronic acid and derivatives thereof;
shellac resin, sandarac gum, dammar resins, elemi gums and copal resins;
deoxyribonucleic acid;
mucopolysaccharides such as chondroitin sulfates; and mixtures thereof.

The film-forming polymer may also be present in the composition in the form of particles dispersed in an aqueous phase, which is generally known as a latex or pseudolatex. The techniques for preparing these dispersions are well known to those skilled in the art.

Aqueous dispersions of film-forming polymer that may be used include the acrylic dispersions sold under the names Neocryl XK-90®, Neocryl A-1070®, Neocryl A-1090®, Neocryl BT-62®, Neocryl A-1079® and Neocryl A-523® by the company Avecia-Neoresins, Dow Latex 432® by the company Dow Chemical, Daitosol 5000 AD® or Daitosol 5000 SJ® by the company Daito Kasey Kogyo; Syntran 5760® by the company Interpolymer, Allianz Opt® by the company Rohm & Haas or the aqueous polyurethane dispersions sold under the names Neorez R-981® and Neorez R-974® by the company Avecia-Neoresins, Avalure UR-405®, Avalure UR-410®, Avalure UR-425®, Avalure UR-450®, Sancure 875®, Avalure UR-445® and Sancure 2060® by the company Noveon, Impranil 85® by the company Bayer, Aquamere H-1511® by the company Hydromer; the sulfopolyesters sold under the brand name Eastman AQ® by the company Eastman Chemical Products, vinyl dispersions, for instance Mexomer PAM®, aqueous polyvinyl acetate dispersions, for instance Vinybran® from the company Nisshin Chemical or those sold by the company Union Carbide, aqueous dispersions of vinylpyrrolidone, dimethylaminopropylmethacrylamide and lauryldimethylpropylmethacrylamidoammonium chloride terpolymer, such as Styleze W from ISP, aqueous dispersions of polyurethane/polyacrylic hybrid polymers such as those sold under the references Hybridur® by the company Air Products or Duromer® from National Starch, and dispersions of core/shell type: for example those sold by the company Atofina under the reference Kynar (core: fluoro; shell: acrylic) or alternatively those described in document U.S. Pat. No. 5,188,899 (core: silica; shell: silicone), and mixtures thereof.

The lipophilic polymer may be in solution or in dispersion in a non-aqueous solvent phase.

Preferably, the first composition comprises at least one water-soluble polymer as defined above.

The compositions according to the present patent application may also contain at least one hydrophilic gelling agent, which may be chosen from:
acrylic or methacrylic acid homopolymers or copolymers or salts and esters thereof and in particular the products sold under the names Versicol F® or Versicol K by the company Allied Colloid, Ultrahold 8® by the company Ciba-Geigy, and polyacrylic acids of Synthalen K type,
the copolymers of acrylic acid and of acrylamide sold in the form of the sodium salt thereof under the name Reten® by the company Hercules and the sodium salts of polyhydroxycarboxylic acids sold under the name Hydagen F® by the company Henkel,
polyacrylic acid/alkyl acrylate copolymers of Pemulen type,
AMPS (polyacrylamidomethylpropanesulfonic acid partially neutralized with aqueous ammonia and highly crosslinked) sold by the company Clariant,
AMPS/acrylamide copolymers of Sepigel® or Simulgel® type sold by the company SEPPIC, and
AMPS/polyoxyethylenated alkyl methacrylate copolymers (crosslinked or non-crosslinked), and mixtures thereof,
associative polyurethanes such as the polymer C₁₆-OE₁₂₀-C₁₆ from the company Servo Delden (sold under the name SER AD FX1100, which is a molecule containing a urethane function and having a weight-average molecular weight of 1300), OE being an oxyethylene unit, Rheolate 205 containing a urea function sold by the company Rheox, or alternatively Rheolate 208 or 204 (these polymers being sold in pure form) or DW 1206B from Rohm & Haas, with a C₂₀ alkyl chain and a urethane bond, sold at 20% active material in water. Solutions or dispersions of these associative polyurethanes, especially in water or in aqueous-alcoholic medium, may also be used. Examples of such polymers that may be mentioned include SER AD FX1010, SER AD FX 1035 and SER AD 1070 from the company Servo Delden, and Rheolate 255, Rheolate 278 and Rheolate 244 sold by the company Rheox. The products DW 1206F and DW 1206J, and also Acrysol RM 184 or Acrysol 44 from the company Rohm & Haas, or Borchigel LW 44 from the company Borchers, may also be used,
and mixtures thereof.

Certain water-soluble film-forming polymers mentioned above may also act as water-soluble gelling agent.

The hydrophilic gelling agents may be present in the compositions according to the invention in a content ranging from 0.05% to 40%, preferably from 0.1% to 20% and better still from 0.5% to 15% by weight relative to the total weight of the composition.

The compositions according to the present patent application may also contain at least one or more oils or organic solvent.

The term “oil or organic solvent” means a non-aqueous substance that is liquid at room temperature and atmospheric pressure. The oil may be volatile or non-volatile.

For the purposes of the invention, the term “volatile oil or organic solvent” means any non-aqueous medium that is capable of evaporating on contact with the keratin materials in less than one hour, at room temperature and atmospheric pressure. The volatile organic solvent(s) and volatile oils of the invention are volatile organic solvents and cosmetic oils that are liquid at room temperature, with a non-zero vapour pressure at room temperature and atmospheric pressure, ranging in particular from 0.13 Pa to 40 000 Pa (10⁻³ to 300 mmHg), in particular ranging from 1.3 Pa to 13 000 Pa (0.01 to 100 mmHg), and more particularly ranging from 1.3 Pa to 1300 Pa (0.01 to 10 mmHg). The term “non-volatile oil” means an oil that remains on the keratin materials at room temperature and atmospheric pressure for at least several hours and that especially has a vapour pressure of less than 10⁻³ mmHg (0.13 Pa).

The oil may be present in the composition in a content ranging from 0.05% to 30% and preferably from 0.1% to 15% by weight relative to the total weight of the composition. The composition according to the invention may comprise volatile oils or non-volatile oils, and mixtures thereof.

The volatile oils (or organic solvents) may be hydrocarbon-based oils, silicone oils or fluoro oils, or mixtures thereof.

The term “hydrocarbon-based oil” means an oil mainly containing hydrogen and carbon atoms and optionally oxygen, nitrogen, sulfur or phosphorus atoms. The volatile hydrocarbon-based oils may be chosen from hydrocarbon-based oils containing from 8 to 16 carbon atoms, and especially branched C₈-C₁₆ alkanes, for instance C₈-C₁₆ isoalkanes of petroleum origin (also known as isoparaffins), for instance isododecane (also known as 2,2,4,4,6-pentamethylheptane), isodecane and isohexadecane, for example the oils sold under the trade names Isopar® or Permethyl®, branched C₈-C₁₆ esters and isohexyl neopentanoate, and mixtures thereof. Other volatile hydrocarbon-based oils, for instance petroleum distillates, especially those sold under the name Shell Solt® by the company Shell, may also be used.

Volatile oils that may also be used include volatile silicones, for instance volatile linear or cyclic silicone oils, especially those with a viscosity 6 centistokes (6×10⁻⁶ m²/s) and especially containing from 3 to 6 silicon atoms, these silicones optionally comprising alkyl or alkoxy groups containing from 1 or 2 carbon atoms. As volatile silicone oils that may be used in the invention, mention may be made especially of octamethylcyclotetrasiloxane, decamethyl-cyclopentasiloxane, dodecamethylcyclohexasiloxane, heptamethylhexyltrisiloxane, heptamethyloctyltri-siloxane, hexamethyldisiloxane, octamethyltrisiloxane, decamethyltetrasiloxane and dodecamethylpentasiloxane, and mixtures thereof.

Volatile organic solvents, especially fluorinated solvents such as nonafluoromethoxybutane or perfluoro-methylcyclopentane, may also be used.

Each of the compositions in accordance with the invention may also comprise at least one non-volatile oil or organic solvent, which may be chosen in particular from non-volatile hydrocarbon-based oils and/or silicone oils and/or fluoro oils.

Non-volatile hydrocarbon-based oils that may especially be mentioned include:
hydrocarbon-based oils of plant origin, such as triglycerides consisting of fatty acid esters of glycerol, the fatty acids of which may have varied chain lengths from C₄ to C₂₄, these chains possibly being linear or branched, and saturated or unsaturated; these oils are especially wheatgerm oil, sunflower oil, grapeseed oil, sesame seed oil, corn oil, apricot oil, castor oil, shea oil, avocado oil, olive oil, soybean oil, sweet almond oil, palm oil, rapeseed oil, cottonseed oil, hazelnut oil, macadamia oil, jojoba oil, alfalfa oil, poppy oil, pumpkin oil, sesame seed oil, marrow oil, rapeseed oil, blackcurrant oil, evening primrose oil, millet oil, barley oil, quinoa oil, rye oil, safflower oil, candlenut oil, passion flower oil and musk rose oil; or alternatively caprylic/capric acid triglycerides such as those sold by the company Stéarineries Dubois or those sold under the names Miglyol 810®, 812® and 818® by the company Dynamit Nobel,
synthetic ethers containing from 10 to 40 carbon atoms;
linear or branched hydrocarbons of mineral or synthetic origin, such as petroleum jelly, polydecenes, hydrogenated polyisobutene such as parleam, and squalane, and mixtures thereof;
synthetic esters such as oils of formula R₁COOR₂ in which R₁ represents a linear or branched fatty acid residue containing from 1 to 40 carbon atoms and R₂ represents an in particular branched hydrocarbon-based chain containing from 1 to 40 carbon atoms, on condition that R₁+R₂≧10, for instance purcellin oil (cetostearyl octanoate), isopropyl myristate, isopropyl palmitate, C₁₂-C₁₅ alkyl benzoate, hexyl laurate, diisopropyl adipate, isononyl isononanoate, 2-ethylhexyl palmitate, isostearyl isostearate, alcohol or polyalcohol octanoates, decanoates or ricinoleates such as propylene glycol dioctanoate; hydroxylated esters such as isostearyl lactate and diisostearyl malate; and pentaerythritol esters;
fatty alcohols that are liquid at room temperature, containing a branched and/or unsaturated carbon-based chain containing from 12 to 26 carbon atoms, for instance octyldodecanol, isostearyl alcohol, oleyl alcohol, 2-hexyldecanol, 2-butyloctanol or 2-undecylpentadecanol;
   higher fatty acids such as oleic acid, linoleic acid or linolenic acid; and mixtures thereof.

The non-volatile silicone oils that may be used in the compositions (i) or (ii) in accordance with the invention may be non-volatile polydimethylsiloxanes (PDMSs), polydimethylsiloxanes comprising alkyl or alkoxy groups, that are pendent and/or at the end of a silicone chain, the groups each containing from 2 to 24 carbon atoms, phenyl silicones, for instance phenyl trimethicones, phenyl dimethicones, phenyltrimethyl-siloxydiphenylsiloxanes, diphenyl dimethicones, diphenylmethyldiphenyltrisiloxanes and 2-phenylethyl trimethylsiloxysilicates.

The fluoro oils that may be used in the compositions in accordance with the invention are, in particular, fluorosilicone oils, fluoro polyethers or fluorosilicones, as described in document EP-A-847 752.

The content of non-volatile oil or organic solvent in the composition in accordance with the invention ranges from 0.01% to 30% by weight, in particular from 0.1% to 25% by weight and better still from 0.1% to 20% by weight relative to the total weight of the composition.

The compositions in accordance with the invention may also comprise at least one dyestuff, for instance pulverulent dyes, liposoluble dyes and water-soluble dyes.

The pulverulent dyestuffs may be chosen from pigments and nacres.

The pigments may be white or coloured, mineral and/or organic, and coated or uncoated. Among the mineral pigments that may be mentioned are titanium dioxide, optionally surface-treated, zirconium oxide, zinc oxide or cerium oxide, and also iron oxide or chromium oxide, manganese violet, ultramarine blue, chromium hydrate and ferric blue. Among the organic pigments that may be mentioned are carbon black, pigments of D&C type, and lakes based on cochineal carmine or on barium, strontium, calcium or aluminium.

The nacres may be chosen from white nacreous pigments such as mica coated with titanium or with bismuth oxychloride, coloured nacreous pigments such as titanium mica with iron oxides, titanium mica with, especially, ferric blue or chromium oxide, titanium mica with an organic pigment of the above-mentioned type, and also nacreous pigments based on bismuth oxychloride.

The liposoluble dyes are, for example, Sudan Red, D&C Red 17, D&C Green 6, (β-carotene, soybean oil, Sudan Brown, D&C Yellow 11, D&C Violet 2, D&C Orange 5, quinoline yellow and annatto.

These dyestuffs may be present in a content ranging from 0.01% to 30% by weight relative to the total weight of the composition.

The compositions in accordance with the invention may also comprise at least one filler.

The fillers may be chosen from those that are well known to those skilled in the art and commonly used in cosmetic compositions. The fillers may be mineral or organic, and lamellar or spherical. Mention may be made of talc, mica, silica, kaolin, polyamide powders, for instance the Nylon® sold under the trade name Orgasol® by the company Atochem, poly-β-alanine powders and polyethylene powders, powders of tetrafluoroethylene polymers, for instance Teflon®, lauroyllysine, starch, boron nitride, expanded polymeric hollow microspheres such as those of polyvinylidene chloride/acrylonitrile, for instance the products sold under the name Expancel® by the company Nobel Industrie, acrylic powders, such as those sold under the name Polytrap® by the company Dow Corning, polymethyl methacrylate particles and silicone resin microbeads (for example Tospearls® from Toshiba), precipitated calcium carbonate, magnesium carbonate and magnesium hydrocarbonate, hydroxyapatite, hollow silica microspheres (Silica Beads® from Maprecos), glass or ceramic microcapsules, metal soaps derived from organic carboxylic acids containing from 8 to 22 carbon atoms and in particular from 12 to 18 carbon atoms, for example zinc, magnesium or lithium stearate, zinc laurate and magnesium myristate.

It is also possible to use a compound that is capable of swelling on heating, and especially heat-expandable particles such as non-expanded microspheres of copolymer of vinylidene chloride/acrylonitrile/methyl methacrylate or of acrylonitrile homopolymer copolymer, for instance those sold, respectively, under the references Expancel® 820 DU 40 and Expancel® 007WU by the company Akzo Nobel.

The fillers may represent from 0.1% to 25% and in particular from 0.2% to 20% by weight relative to the total weight of the composition.

The compositions in accordance with the invention may also comprise fibres that allow an improvement in the lengthening effect.

The term “fibre” should be understood as meaning an object of length L and diameter D such that L is very much greater than D, D being the diameter of the circle in which the cross section of the fibre is inscribed. In particular, the ratio L/D (or shape factor) is chosen in the range from 3.5 to 2500, especially from 5 to 500 and in particular from 5 to 150.

The fibres that may be used in the composition of the invention may be mineral or organic fibres of synthetic or natural origin. They may be short or long, individual or organized, for example braided, and hollow or solid. They may have any shape, and may especially have a circular or polygonal (square, hexagonal or octagonal) cross section, depending on the intended specific application. In particular, their ends are blunt and/or polished to prevent injury.

In particular, the fibres have a length ranging from 1 μm to 10 mm, preferably from 0.1 mm to 5 mm and better still from 0.3 mm to 3.5 mm. Their cross section may be within a circle of diameter ranging from 2 nm to 500 μm, preferably ranging from 100 nm to 100 μm and better still from 1 μm to 50 μm. The weight or yarn count of the fibres is often given in denier or decitex, and represents the weight in grams per 9 km of yarn. In particular, the fibres according to the invention may have a yarn count chosen in the range from 0.15 to 30 denier and better still from 0.18 to 18 denier.

The fibres that may be used in the composition of the invention may be chosen from rigid or non-rigid fibres, and may be of synthetic or natural, mineral or organic origin.

Moreover, the fibres may or may not be surface-treated, may be coated or uncoated, and may be coloured or uncolored.

As fibres that may be used in the composition according to the invention, mention may be made of non-rigid ® fibres such as polyamide (Nylon®) fibres or rigid fibres such as polyimideamide fibres, for instance those sold under the names Kermel® and Kermel® Tech by the company Rhodia or poly(p-phenyleneterephthalamide) (or aramid) fibres sold especially under the name Kevlar® by the company DuPont de Nemours.

The fibres may be present in the composition according to the invention in a content ranging from 0.01% to 10% by weight, in particular from 0.1% to 5% by weight and more particularly from 0.3% to 3% by weight relative to the total weight of the composition comprising them.

The compositions in accordance with the invention may also comprise at least one cosmetic active agent.

As cosmetic active agents that may be used in the compositions in accordance with the invention, mention may be made especially of antioxidants, preserving agents, fragrances, neutralizers, emollients, thickeners, coalescers, plasticizers, moisturizers, vitamins and screening agents, in particular sunscreens, and mixtures thereof.

The first and/or second composition may be in liquid, semi-liquid, pasty or solid form.

Needless to say, a person skilled in the art will take care to select the optional additional additives and/or the amount thereof such that the advantageous properties of the composition according to the invention are not, or are not substantially, adversely affected by the envisaged addition, and especially such that they do not interfere with the complexation reaction between the alginic acid-based compound and the complexing agent.

A subject of the invention is also a cosmetic process for coating keratin materials, which consists in applying to the said keratin materials:
at least one coat of at least one first composition comprising an aqueous phase, at least one alginic acid-based compound A and at least one compound B in the form of a water-soluble salt, the said compound being capable of releasing a gas on contact with an acid having a pKa ranging from 1 to 6, and then
at least one coat of at least one second composition comprising at least one acid having a pKa ranging from 1 to 6 and at least one agent C for complexing the alginic acid-based compound, the said agent being in the form of a water-soluble salt.

The first and second compositions are as defined above.

The above eyelash coating process may comprise a step of heating the first and/or second compositions, prior to, simultaneously with or subsequently to their application to the eyelashes.

According to one embodiment, the eyelash coating process consists in applying to the eyelashes:
at least one coat of at least one first composition comprising an aqueous phase, at least one alginic acid-based compound A and at least one compound B in the form of a water-soluble salt, the said compound being capable of releasing a gas on contact with an acid having a pKa ranging from 1 to 6, and then
at least one coat of at least one second composition comprising at least one acid having a pKa ranging from 1 to 6 and at least one agent C for complexing the alginic acid-based compound, the said agent being in the form of a water-soluble salt, and then
in subjecting the film obtained after application of the first and second compositions to a source of heat.

The source of heat may be, for example, a heating applicator.

### Makeup Removal

The water-insoluble film obtained by reaction between the alginic acid-based compound of the first composition described above and the complexing agent of the second composition described above may be readily removed using a sequestrant (also known as a chelating agent) for the agent for complexing the alginic acid-based compound.

Accordingly, the invention also relates to a process for removing and/or cleansing away makeup films formed by the application to keratin materials of a first composition comprising an aqueous phase, at least one alginic acid-based compound and at least one compound in the form of a water-soluble salt, the said compound being capable of releasing a gas on contact with an acid having a pKa ranging from 1 to 6, and of a second composition comprising at least one acid having a pKa ranging from 1 to 6 and at least one agent for complexing the alginic acid-based compound, the said agent being in the form of a water-soluble salt, the process comprising at least one step of applying onto the said makeup films a makeup-removing and/or cleansing composition comprising, in a physiologically acceptable medium, an aqueous phase and at least one sequestrant for the said complexing agent.

After applying the makeup-removing and/or cleansing composition to the makeup film obtained after application of the first and second compositions, the sequestrant binds with the complexing agent (in particular the multivalent ions) present in the complex of complexing agent-alginic acid-based compound, which leads to dissociation of the said complex and disintegration of the makeup film on the eyelashes.

A subject of the invention is also a cosmetic kit comprising:
a first composition comprising an aqueous phase, at least one alginic acid-based compound A and at least one compound B in the form of a water-soluble salt, the said compound being capable of releasing a gas on contact with an acid having a pKa ranging from 1 to 6,
a second composition comprising at least one acid having a pKa ranging from 1 to 6 and at least one agent C for complexing the alginic acid-based compound, the said agent being in the form of a water-soluble salt, and
a makeup-removing and/or cleansing composition comprising, in a physiologically acceptable medium, an aqueous phase and at least one sequestrant for the said complexing agent.

The first and second compositions are as described above.

The sequestrant for the complexing agent is chosen in particular from carboxylic acids other than the acid having a pKa ranging from 1 to 6 present in the second composition, preferably aminocarboxylic acids, phosphonic acids, preferably aminophosphonic acids, polyphosphoric acids, preferably linear polyphosphoric acids, and salts and derivatives thereof.

The salts are especially alkali metal, alkaline-earth metal, ammonium and substituted ammonium salts.

The chelating agents may be chosen in particular from:
aminocarboxylic acids (i.e. acids comprising at least one carboxylic acid group) such as the compounds having the following INCI name:
   phytic acid,
   diethylenetriaminepentaacetic acid (DTPA),
   ethylenediaminedisuccinic acid (EDDS) and trisodium ethylenediamine disuccinate such as Octaquest E30 from Octel,
   ethylenediaminetetraacetic acid (EDTA),
   ethylenediamine-N,N′-diglutaric acid (EDDG),
   glycinamide-N,N′-disuccinic acid (GADS),
   2-hydroxypropylenediamine-N,N′-disuccinic acid (HPDDS),
   ethylenediamine-N,N′-bis(ortho-hydroxyphenylacetic acid) (EDDHA),
   N,N′-bis(2-hydroxybenzyl)ethylenediamine-N,N′-diacetic acid (HBED),
   nitrilotriacetic acid (NTA),
   methylglycine diacetic acid (MGDA),
   N-2-hydroxyethyl-N,N-diacetic acid and glyceryl imino diacetic acid (as described in documents EP-A-317 542 and EP-A-399 133),
   iminodiacetic acid-N-2-hydroxypropyl sulfonic acid and aspartic acid N-carboxymethyl N-2-hydroxypropyl-3-sulfonic acid (as described in EP-A-516 102),
   beta-alanine-N,N′-diacetic acid, aspartic acid-N,N′-diacetic acid and aspartic acid-N-monoacetic acid (described in EP-A-509 382),
   chelating agents based on iminodisuccinic acid (IDSA) (as described in EP-A-509 382), ethanoldiglycine acid,
   phosphonobutane tricarboxylic acid, such as the compound sold by Bayer under the reference Bayhibit AM,
   tetrasodium glutamate diacetate (GLDA) such as Dissolvine GL38 or 45S from Akzo Nobel,
chelating agents based on mono- or polyphosphonic acid, such as the compounds having the following INCI name:
   diethylenetriaminepenta (methylenephosphonic acid) (DTPMP),
   ethane-1-hydroxy-1,1,2-triphosphonic acid (E1HTP),
   ethane-2-hydroxy-1,1,2-triphosphonic acid (E2HTP),
   ethane-1-hydroxy-1,1-diphosphonic acid (EHDP),
   ethane-1,1,2-triphosphonic acid (ETP),
   ethylenediaminetetramethylenephosphonic acid (EDTMP),
   hydroxyethane-1,1-diphosphonic acid (HEDP),
chelating agents based on polyphosphoric acid, such as the compounds having the following INCI name:
   sodium tripolyphosphate (STP),
   tetrasodium diphosphate,
   hexametaphosphoric acid,
   sodium metaphosphate,
   phytic acid,
      salts and derivatives thereof,
      and mixtures thereof.

Mention may also be made of carboxylated derivatives of fructan inulin (for instance those described in document U.S. Pat. No. 6,280,628).

Among the chelating agents mentioned, ethylenediamine-tetraacetic acid (EDTA), diethylenetriaminepentaacetic acid (DTPA), S,S′-ethylenediaminedisuccinic acid (EDDS), ethylenediaminetetramethylenephosphonic acid (EDTMP), phosphonobutanetricarboxylic acid and tetrasodium glutamate diacetate (GLDA), and mixtures thereof, are preferably used.

The sequestrant may be present in a content ranging from 0.01% to 30% by weight, preferably from 0.05% to 15% by weight and better still from 0.1% to 10% by weight relative to the total weight of the makeup-removing and/or cleansing composition.

Preferably, the sequestrant is present in the form of an aqueous solution, i.e. the makeup-removing and/or cleansing composition comprises an aqueous phase as defined above.

The compositions according to the invention may each be conditioned in a container delimiting at least one compartment that comprises the said composition, the said container being closed by a closing member.

The container is preferably associated with an applicator, especially in the form of a brush comprising an arrangement of bristles maintained by a twisted wire. Such a twisted brush is especially described in U.S. Pat. No. 4,887,622. It may also be in the form of a comb comprising a plurality of application members, obtained especially by moulding. Such combs are described, for example, in patent FR 2 796 529. The applicator may be solidly attached to the container, as described, for example, in patent FR 2 761 959. Advantageously, the applicator is solidly attached to a stem, which is itself solidly attached to the closing member.

It may be a moulded comb or a moulded brush as described in documents WO 06/125 122 or FR 2 769 529.

The closing member may be coupled to the container by screwing. Alternatively, the coupling between the closing member and the container takes place other than by screwing, especially via a bayonet mechanism, by click-fastening or by tightening. The term “click-fastening” in particular means any system involving the passing of a rim or bead of material by elastic deformation of a portion, especially of the closing member, followed by return to the elastically unstressed position of the said portion after the rim or bead has been passed.

The container may be at least partly made of thermoplastic material. Examples of thermoplastic materials that may be mentioned include polypropylene and polyethylene.

Alternatively, the container is made of a non-thermoplastic material, especially of glass or metal (or alloy).

The container is preferably equipped with a drainer located in the region of the aperture of the container. Such a drainer makes it possible to wipe the applicator and, optionally, the stem to which it may be solidly attached. Such a drainer is described, for example, in patent FR 2 792 618.

Preferably, the first composition and the second composition are conditioned in separate conditioning.

Each composition may be conditioned separately in the same conditioning article. Each composition may also be conditioned in a compartment within the same conditioning article, the mixing of the two compositions being performed at the extremity(ies) of the conditioning article during the expulsion of each composition.

Alternatively, each of the first and second compositions may be conditioned in a separate conditioning article.

The examples that follow are given as illustrations of the present invention, and shall not in any way limit the scope thereof.

### EXAMPLES

The following compositions were prepared. The amounts indicated are expressed as mass percentages relative to the total weight of the composition.

### Composition 1

| | |
|---|---|
| Sodium alginate (Kelcosol from ISP) | 3% |
| Sodium bicarbonate (Socal 90 A from Solvay) | 1% |
| Preserving agents | qs |
| Water | qs 100 |

### Composition 2

| | |
|---|---|
| Tartaric acid | 3% |
| Calcium chloride dihydrate (Merck) | 3% |
| Water | qs 100 |

A coat of the first composition is applied to the eyelashes, onto which is applied a coat of the second composition.

After a few seconds, swelling of the film on the eyelashes is observed, due to the release of CO₂ bubbles trapped in the alginate gel complexed with the Ca²⁺, which leads to a volumizing effect on the eyelashes.

After drying, the deposit obtained on the eyelashes is an alveolar structure.

1. A keratin material coating kit comprising:
a first composition comprising an aqueous phase, at least one alginic acid-based compound A and at least one compound B in the form of a water-soluble salt, the said compound B being capable of releasing a gas on contact with an acid having a pKa ranging from 1 to 6, and
a second composition comprising at least one acid having a pKa ranging from 1 to 6 and at least one agent C for complexing the alginic acid-based compound, the said agent being in the form of a water-soluble salt.

2. The kit according to claim 1, wherein a weight-average molecular mass of the alginic acid-based compound is from 10 000 to 1 000 000.

3. The kit according to claim 1, wherein the alginic acid-based compound is a sodium or potassium alginate.

4. The kit according to claim 1, wherein a content of the alginic acid-based compound is from 0.1% to 30% by weight relative to the total weight of the first composition comprising it.

5. The kit according to claim 1, wherein a content of the aqueous phase is from 1% to 95% by weight relative to the total weight of the composition.

6. The kit according to claim 1, wherein the compound B which is capable of releasing a gas is an alkali metal carbonate.

7. The kit according to claim 1, wherein the compound B is sodium carbonate or sodium bicarbonate.

8. The kit according to claim 7, wherein the compound B is sodium bicarbonate.

9. The kit according to claim 1, wherein a content of the compound B is from 0.01% to 30% by weight relative to the total weight of the first composition.

10. The kit according to claim 1, wherein the agent C for complexing the alginic acid-based compound is a water-soluble salt selected from the group consisting of polyelectrolytes and multivalent ions, and salts thereof.

11. The kit according to claim 10, wherein the agent for complexing the alginic acid-based compound is a salt of a multivalent cation or of a polycation.

12. The kit according to claim 11, wherein the cation is a divalent cation selected from the cations of the group consisting of calcium, magnesium, aluminium, barium, zinc, nickel, copper (+II), manganese and mixtures thereof.

13. The kit according to claim 1, wherein the agent for complexing the alginic acid-based compound is calcium chloride.

14. The kit according to claim 1, wherein a content of the complexing agent C is from 0.01% to 30% by weight relative to the total weight of the second composition.

15. The kit according to claim 1, wherein the acid having a pKa ranging from 1 to 6 in the second composition is a sulfuric acid acids, a carboxylic acid or a mixture thereof.

16. The kit according to claim 1, wherein the acid having a pKa ranging from 1 to 6 in the second composition is tartaric acid or citric acid.

17. The kit according to claim 1, wherein a content of the acid having a pKa ranging from 1 to 6 is from 0.0001% to 15% by weight relative to the total weight of the second composition.

18. A cosmetic process for coating keratin materials, comprising applying to the said keratin materials:
at least one coat of at least one first composition comprising an aqueous phase, at least one alginic acid-based compound A and at least one compound B in the form of a water-soluble salt, the said compound being capable of releasing a gas on contact with an acid having a pKa ranging from 1 to 6, and
th then applying
at least one coat of at least one second composition comprising at least one acid having a pKa ranging from 1 to 6 and at least one agent C for complexing the alginic acid-based compound, the said agent being in the form of a water-soluble salt.

19. A process for removing and/or cleansing away makeup films formed by the application, to keratin materials, of a first composition comprising an aqueous phase, at least one alginic acid-based compound A and at least one compound B in the form of a water-soluble salt, the said compound being capable of releasing a gas on contact with an acid having a pKa ranging from 1 to 6, and of a second composition comprising at least one acid having a pKa ranging from 1 to 6 and at least one agent C for complexing the alginic acid-based compound, the said agent being in the form of a water-soluble salt, comprising:
applying onto the said makeup films a makeup-removing and/or cleansing composition comprising, in a physiologically acceptable medium, an aqueous phase and at least one sequestrant for the said complexing agent.

20. The process according to claim 19, wherein the sequestrant for the complexing agent is selected from the group consisting of aminocarboxylic acids, phosphonic acids, aminophosphonic acids, polyphosphoric acids, salts and derivatives thereof.

21. The process according to claim 19 wherein the sequestrant for the complexing agent is selected from the group consisting of:
diethylenetriaminepentaacetic acid (DTPA),
ethylenediaminedisuccinic acid (EDDS) and trisodium ethylenediamine disuccinate,
ethylenediaminetetraacetic acid (EDTA),
ethylenediamine-N,N′-diglutaric acid (EDDG),
glycinamide-N,N′-disuccinic acid (GADS),
2-hydroxypropylenediamine-N,N′-disuccinic acid (HPDDS),
ethylenediamine-N,N′-bis(ortho-hydroxyphenylacetic acid) (EDDHA),
N,N′-bis(2-hydroxybenzyl)ethylenediamine-N,N′-diacetic acid (HBED),
nitrilotriacetic acid (NTA),
methylglycinediacetic acid (MGDA),
N-2-hydroxyethyl-N,N-diacetic acid and glyceryl iminodiacetic acid,
iminodiacetic acid-N-2-hydroxypropyl sulfonic acid and aspartic acid N-carboxymethyl N-2-hydroxypropyl-3-sulfonic acid,
beta-alanine-N,N′-diacetic acid, aspartic acid-N,N′-diacetic acid and aspartic acid-N-monoacetic acid,
ethanoldiglycine acid,
phosphonobutanetricarboxylic acid,
tetrasodium glutamate diacetate (GLDA),
chelating agents based on mono- or polyphosphonic acid, such as:
diethylenetriaminepenta(methylenephosphonic acid) (DTPMP),
ethane-1-hydroxy-1,1,2-triphosphonic acid (E1 HTP),
ethane-2-hydroxy-1,1,2-triphosphonic acid (E2HTP),
ethane-1-hydroxy-1,1-diphosphonic acid (EHDP),
ethane-1,1,2-triphosphonic acid (ETP),
ethylenediaminetetramethylenephosphonic acid (EDTMP),
hydroxyethane-1,1-diphosphonic acid (HEDP),
sodium tripolyphosphate (STP),
tetrasodium diphosphate,
hexametaphosphoric acid,
sodium metaphosphate,
phytic acid,
salts and derivatives thereof,
and mixtures thereof.

22. The process according to claim 19, wherein the sequestrant is selected from ethylenediaminetetraacetic acid (EDTA),
diethylenetriaminepentaacetic acid (DTPA), S,S′-ethylenediaminedisuccinic acid (EDDS), ethylenediaminetetramethylenephosphonic acid (EDTMP),
phosphonobutanetricarboxylic acid, tetrasodium glutamate diacetate (GLDA), phytic acid, and salts, derivatives or mixtures thereof.

23. The process according to claim 22, wherein the sequestrant is ethylenediaminetetraacetic acid.

24. The process according to claim 19, wherein a content of the sequestrant is from 0.01% to 30% by weight relative to the total weight of the makeup-removing and/or cleansing composition.

25. A cosmetic kit comprising:
a first composition comprising an aqueous phase, at least one alginic acid-based compound A and at least one compound B in the form of a water-soluble salt, the said compound being capable of releasing a gas on contact with an acid having a pKa ranging from 1 to 6,
a second composition comprising at least one acid having a pKa ranging from 1 to 6 and at least one agent C for complexing the alginic acid-based compound, the said agent being in the form of a water-soluble salt, and
a makeup-removing and/or cleansing composition comprising, in a physiologically acceptable medium, an aqueous phase and at least one sequestrant for the said complexing agent.
